(19) United States
(12) Patent Application Publication
(10) Pub. No.: US 2011/0071632 A1
(43) Pub. Date: Mar. 24, 2011

(54) MODULUS-MATCHING GRAFT

(76) Inventors: SOMASUNDAR SUDHANSHU, San Diego, CA (US); Mahar Andrew, San Diego, CA (US)

(21) Appl. No.: 12/703,649

(22) Filed: Feb. 10, 2010

Related U.S. Application Data
(60) Provisional application No. 61/151,492, filed on Feb. 10, 2009.

(51) Int. Cl. A61F 2/28

(52) U.S. Cl. 623/16.11

(57) ABSTRACT

Methods and systems for matching a bone graft's material properties to that of a patient's recipient bone are provided to improve the interaction between graft bone and recipient bone and to optimize the performance of the graft over time. A method of matching properties of a recipient bone with properties of a bone graft is provided. The method includes obtaining data on material properties of a recipient bone; comparing the material properties of the recipient bone to various modified bone grafts having differing range of values for the material properties; and selecting a modified bone graft having a range of values into which the material properties of the recipient bone falls within. The material properties may include yield strength, ultimate strength (tensile, compressive, and shear), modulus of elasticity, Young's Modulus, compression modulus, bulk modulus, rigidity modulus, shear modulus, compressive strength, resilience, toughness, stiffness, elastic energy, plastic energy, strain energy, and hysteresis.

### CROSS-REFERENCES TO RELATED APPLICATIONS

This application also claims priority to U.S. Provisional Application No. 61/151,492, filed on Feb. 10, 2009, entitled “MODULUS-MATCHING GRAFT”, the contents of which is incorporated herein by reference in its entirety.

### BACKGROUND OF THE INVENTION

1. Field of the Invention

The present invention relates generally to medical implants and more particularly to methods and systems for matching medical implant material properties to that of patient's bone characteristics.

2. Description of the Related Art

Bone grafting refers to a wide variety of medical and dental surgical procedures by which the formation of new bone in a patient is augmented or stimulated. Bone grafting is used in many types of orthopedic procedures to treat bone fractures or loss, to repair injured bone that has not healed, and to fuse together joints to prevent movement. With particular reference to the spine, grafts have been used to stabilize the spine and to prevent movement by selected vertebral segments, which may be a significant cause of pain in some patients. Grafts have also been used to correct or stop the progress of spinal deformity, such as scoliosis, and to provide structural support for fractures of the spine.

Suitable grafts can be harvested from bones in the patient's own body (autografts), from bones in members of the same species (allograft), and from bones in members of other animal species (xenograft). Alternatively, bone grafts can be created from a wide variety of natural and/or synthetic materials, such as collagen, polymers, hydroxyapatite, calcium sulfate, ceramics, biocompatible metals, and bioresorbable polymers, among many others. It is understood that bone grafts can include those which have a predetermined shaped or which are comprised of smaller particles that can be formed into a desired shape at the time of implantation.

Bone grafts are commonly implanted into fragile, weak, or brittle bones. Osteoporotic bones, for example, suffer from reduced bone mineral density and increased risk of fracture. As a result, conventional bone grafts are frequently stiffer than the recipient bone into which they are implanted. In some cases, there may be significant differences between the modulus of elasticity, modulus of compression, and other material properties of the conventional graft and that of the recipient bone. Moreover, this mismatched graft bone-recipient bone interface may increase the risk of fracture, implant subsidence, and even failure in the recipient bone over time. Thus, the ability to match the modulus of elasticity, modulus of compression, and other material properties of a bone graft and a patient's bone remains a significant aspect in tissue engineering.

### SUMMARY OF THE INVENTION

Methods and systems are disclosed herein for matching a bone graft's material properties to that of a recipient patient's bone to improve the interaction between graft bone and recipient bone and to optimize the performance of the graft over time.

In a first aspect, embodiments of the present invention provide a method of modifying the properties of a bone graft to match the properties of recipient bone, the method comprising obtaining data on one or more material properties of a recipient bone; comparing the material properties of the recipient bone to various modified bone grafts having differing range of values for the one or more material properties; and selecting a modified bone graft having a range of values into which the one or more material properties of the recipient bone falls within.

In another aspect, embodiments of the present invention provide a method of modifying the properties of a bone graft to match the properties of recipient bone, the method comprising obtaining data on properties of a recipient bone; modifying a bone graft to match the properties of the recipient bone; and testing the bone graft to confirm the properties of the bone graft match the properties of the recipient bone.

In another aspect, embodiments of the present invention provide a method of modifying the properties of a bone graft to match the properties of recipient bone, the method comprising modifying a material property of a first bone graft, where the material property falls within a first range of values; modifying the same material property of a second bone graft, where the material property falls within a second, non-overlapping range of values; obtaining data on the same material property of a recipient bone; and choosing the first or second bone graft, such that the material property of the recipient bone falls within the range of material property values of the chosen bone graft.

In many embodiments, the modified properties are material properties, including yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis.

In many embodiments, the modified properties are compositional properties, including bone mass density and bone calcium percentage.

In many embodiments, the preferred embodiment, modifying the bone graft includes demineralizing the bone graft using an acid wash.

In another aspect, embodiments of the present invention provide a bone graft kit comprising a modified bone graft and a vacuum sealed package, where the modified bone graft is sealed inside the package.

In another aspect, embodiments of the present invention provide a method of treating an osteoporotic patient comprising identifying an osteoporotic patient; obtaining a bone graft; modifying the bone graft to more closely match the patient's bone characteristics; and implanting the bone graft into the osteoporotic patient.

Other objects, features and advantages of the present invention will become apparent to those skilled in the art from the following detailed description.

### BRIEF DESCRIPTION OF THE DRAWINGS

FIG. 1 is a flow chart depicting an embodiment of a medical implant modulus-matching process.

FIG. 2 is a stress-strain curve of one embodiment of a modulus-matched medical implant.

FIG. 3 is a flow chart depicting another embodiment of a medical implant modulus-matching process.

FIG. 4 is compares stress and strain values for embodiments of modulus-matched medical implants and unmodified medical implants.

FIG. 5 compares stress and strain values for additional embodiments of modulus-matched medical implants and unmodified medical implants.

FIG. 6 illustrates material property values of embodiments of modulus-matched medical implants and unmodified medical implants referenced in FIGS. 4 and 5.

FIG. 7 compares modulus of compression, resilience, toughness, yield strength, and ultimate strength for embodiments of modulus-matched medical implants and unmodified medical implants referenced in FIGS. 4 through 6.

FIG. 8 compares force and displacement values for embodiments of modulus-matched medical implants and unmodified medical implants.

FIG. 9 compares force and displacement values for additional embodiments of modulus-matched medical implants and unmodified medical implants.

FIG. 10 illustrates material property values of embodiments of modulus-matched medical implants and unmodified medical implants referenced in FIGS. 8 and 9.

FIG. 11 compares stiffness and modulus of compression for embodiments of modulus-matched medical implants and unmodified medical implants referenced in FIGS. 8 through 10.

FIG. 12 compares resilience, toughness, yield strength, and ultimate strength for embodiments of modulus-matched medical implants and unmodified medical implants referenced in FIGS. 8 through 10.

FIG. 14 compares yield strength and ultimate strength for embodiments of modulus-matched medical implants and unmodified medical implants referenced in FIGS. 4 through 12.

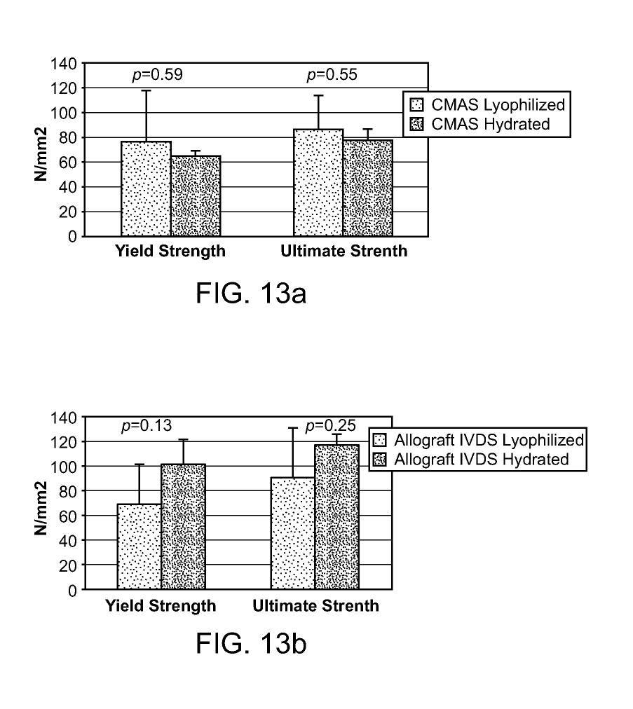

### DETAILED DESCRIPTION OF EMBODIMENTS OF THE INVENTION

The present disclosure is directed to methods and systems for matching the properties of a bone graft to the properties of an individual patient's bone to optimize the interaction between two ordinarily dissimilar materials. Although the present disclosure describes the methods and systems for modulus matching medical grafts, particularly bone grafts, it is understood that the methods and systems can also be applied for a wide variety of medical and dental applications and also soft tissue applications, such as in regenerative medicine and tissue engineering. Accordingly, the term “graft” as used herein can be comprised of any naturally occurring tissue including bone tissue and soft tissues as well as any non-naturally occurring substance used as a graft, or any combination thereof.

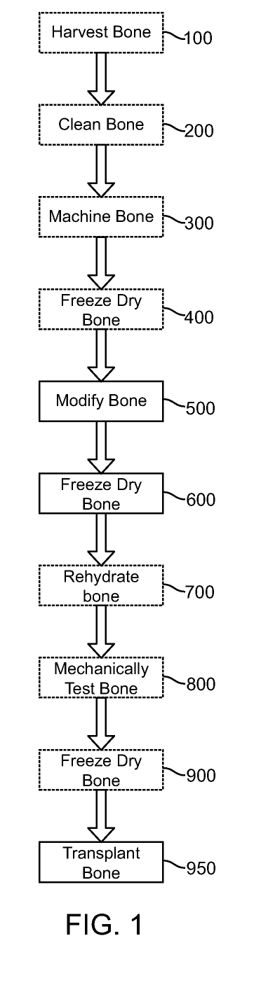

Systems and methods to alter the material characteristics of a bone graft to more closely match those of a recipient patient's bone will now be described with reference to the flow chart depicted in FIG. 1. At step 100, a portion of a recipient's bone or a portion of a donor bone is harvested. It will be understood the methods described herein can be used with grafts harvested from bones in the patient's own body as well as with grafts harvested from a human donor or members of another species. In preferred embodiments, bone is harvested from a healthy human donor.

At step 200, the harvested bone is cleaned and prepared for implantation in a patient in need of a bone graft. The cleaning and preparation of the bone can be accomplished in accordance with conventional techniques, such as, for example, AATB and FDA standards. It will be understood that the systems and methods described herein can be used in patients with healthy bones as well as with weak or brittle bones, such as osteoporotic bones susceptible to fracture. Persons of skill will also understand the systems and methods described herein can be used to treat any condition afflicted by suboptimal interaction between graft bone and recipient bone. Such conditions include but are not limited to osteoporosis, aging spine pathologies, scoliosis, spinal degeneration, spondylolysis, spondylolisthesis, tumor, trauma, and infection.

Continuing to step 300, the cleaned bone is machined into a desired graft shape, such as but not limited to a generally cylindrical, c-shaped, square, oval, or rectangular shape. In some preferred embodiments, the donor bone is machined into a conventional shape for spinal intervention, including but not limited to an anterior cervical cage, FLIP TLIF, ALIF, or XLIF configurations.

Moving to optional step 400, the donor bone is optionally frozen, or more preferably, freeze-dried. Medical implants, such as bone grafts, are commonly dehydrated and freeze-dried for storage prior to use or implantation. Freeze-drying involves a freezing process under negative pressure that results in a graft having low residual moisture. One advantage of this process is that it allows for storage of bone grafts and other biological material at room temperature. It also provides for increased shelf-life with reduced biochemical changes to the bone graft. In some embodiments, the donor bone is harvested, cleaned, machined, then frozen or freeze-dried at one location, such as but not limited to a tissue bank, hospital, or clinic, then transported to a second location for modulus-matching, described in further detail below. In other embodiments, the donor bone is harvested, cleaned, then frozen or freeze-dried at one location, then machined and modulus-matched at a second location. Freezing or freeze-drying a graft thus offers the advantage of easily transporting the graft between locations, and provides easy and economical storage prior to use.

Turning now to step 500, the material properties of the bone graft are altered to match those of the recipient patient's bone. In some embodiments, the properties of the bone graft are altered by a process of demineralization. Demineralization can be accomplished via chemical processing, including but not limited to subjecting the bone graft to an acid wash, chelating agents, or electrolysis. In some embodiments, the bone graft is placed on an orbital shaker and demineralized in a strongly acidic solution, such as but not limited to a half-normal (0.5 N) solution of hydrochloric acid for approximately 1 minute, 5 minutes, 30 minutes, 1 hour, 2 hours, 4 hours, 8 hours, 20 hours, or 50 hours. Persons of skill in the art will understand that the bone graft can be modified and/or subjected to an acid wash using conventional containers, such as but not limited to a pressurized container, vacuum container, vacuum pressure container, pressure cycle container, or orbital shaker. In some embodiments, the bone graft is demineralized for approximately 8 hours. Persons of skill in the art will understand that the duration of the acid wash can be varied to achieve specific, desired material properties in the modified bone graft. Specific material properties that can be matched in accordance with embodiments of the present invention are described in greater detail below with reference to step 800.

Without being bound to any particular theory, it is believed that demineralizing the bone graft alters the surface properties of the bone graft to better match the material properties of the patient's recipient bone. Further, it is believed that subjecting the bone graft to varying acid concentrations and exposure times can alter the characteristics of the bone graft as a whole, not just the surface properties of the bone graft.

The modified bone graft is next frozen or, more preferably, freeze-dried at step 600. As described above, freeze-drying results in a graft having low residual moisture, allowing for storage at room temperature and easy transportation between locations.

At optional step 700, the bone graft is rehydrated before mechanical testing is performed at step 800. Bone grafts are typically rehydrated or reconstituted with a solution of water, saline, bone marrow, blood, or protein prior to implantation in a patient or recipient.

Rehydration of freeze-dried bone grafts typically involves soaking the grafts in a solution until the grafts reach the desired level of hydration. Depending on the size of the graft, among other factors, rehydration and reconstitution of a bone graft can take anywhere from one minute to several hours. Smaller grafts may take less time to rehydrate, as little as one to two minutes for example. Depending on the size and characteristics of a graft, two to three hours may be required to rehydrate the graft. One recommended reconstitution time is approximately 20 minutes. Systems and methods to expeditiously reconstitute and hydrate medical grafts and to effectively and uniformly seed the medical grafts with biological components and cells are described in U.S. patent application Ser. No. 12/251,297, which is hereby incorporated by reference in its entirety.

In some embodiments, the bone graft is rehydrated for approximately twenty minutes, a typical duration for bone graft rehydration. In other embodiments, the bone graft is soaked in water, saline, bone marrow, blood, or protein for between approximately thirty seconds, 1 minute, 5 minutes, 10 minutes, 15 minutes, 20 minutes, 25 minutes, 30 minutes, 60 minutes, up to 90 minutes.

Following rehydration, mechanical testing can optionally be performed on the bone graft at step 800 to obtain data on the material properties of the bone graft. A comparison of this bone graft data to corresponding data from the patient's recipient bone can verify the material properties of the two portions of bone are an optimized or improved match. Examples of material properties that can be tested and/or optimized using the systems and methods described herein include the yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis of the bone graft. Compositional characteristics that can be tested and/or optimized include, but are not limited to, bone mass density (BMD), bone calcium percentage, and hard mineral content. Persons of skill in the art will understand one or a combination of characteristics can be chosen to be optimized in the bone graft. In some embodiments, modulus of elasticity, modulus of compression, resilience, toughness, and stiffness can be optimized in the bone graft using the methods described herein, while preserving the bone graft's original yield strength and ultimate strength.

Persons of skill in the art will understand that the systems and methods for modulus matching grafts as described herein need not include mechanical testing of a patient's bone or a bone graft. As described above, a database or other information-gathering system can be developed in order to determine that specific modifications to bone grafts yield specific desired material characteristics. In such an embodiment, a modulus-matched graft can be selected for implantation into a patient based on known patient factors, patient bone characteristics, and/or known characteristics of modified bone grafts. In the alternative, mechanical testing may be employed when a custom-modified bone graft, as opposed to an “off-the-shelf” modified graft, is desired. In such an embodiment, a sample of the custom-modified bone graft can be mechanically tested to ensure the material properties of the modified bone graft match those of the patient's bone.

Mechanical testing of the modified bone graft can be performed in accordance with technical industry standards developed by, for example, ASTM International or the International Organization for Standardization (ISO). Such standards can be found in various published ASTM Standard Specifications and Test Methods and ISO Standards. ASTM Standard F2077-03, for example, provides test methods for intervertebral body fusion devices and is hereby incorporated by reference.

Mechanical Testing

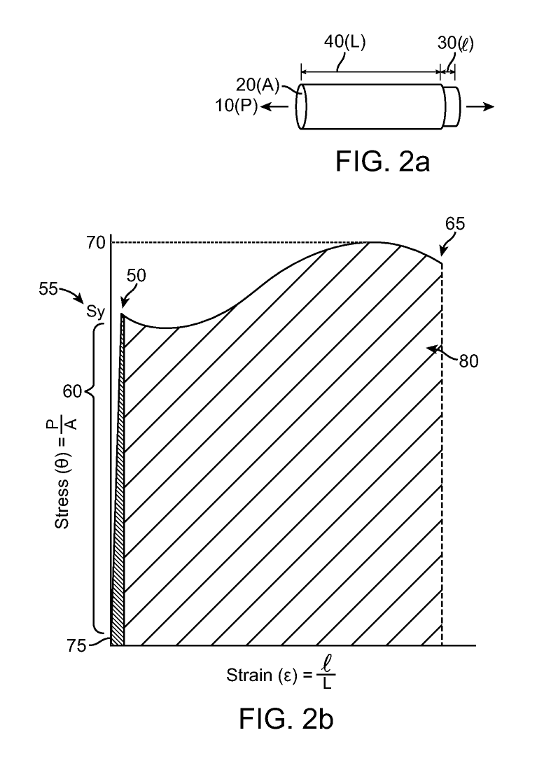

While any of a number of characteristics described above can be optimized using the methods described herein, testing to determine a bone graft's yield strength, ultimate strength, modulus of elasticity, modulus of compression, resilience, and toughness will now be described with reference to the stress-strain curve illustrated in FIG. 2. Mechanical testing of a material yields a specific stress-strain curve for that specific material. Here, the material can be the modified bone graft, a sample of the modified bone graft, or, as will be described in more detail below, a sample of the patient's recipient bone. The stress-strain curve is a graphical representation of the relationship between stress and strain occurring in the material during testing. Stress values are derived by measuring the load applied on the material, while strain values are derived from measuring the deformation of the material as a result of the applied load. Deformation of the sample can include elongation, compression, torsion, bending, shear, or distortion.

More specifically, stress is calculated by dividing the uniaxial load P (10) applied to the material by the surface area A (20) over which the load is applied. Persons of skill in the art will understand that a stress-strain curve such as that illustrated in FIG. 2 can be the result of a tensile test, where P is a uniaxial tensile load, or the result of a compression test, where P is a uniaxial compressive load. Strain is calculated by dividing the deformation, or change in length 1 of the sample (30), by the initial length L of the sample (40) before loading. As illustrated in FIG. 2, stress values are represented along the y-axis while strain values are represented along the x-axis.

Persons of skill will understand the nature of the stress-strain curve varies from material to material, and from sample to sample. Thus, unmodified bone grafts will exhibit stress-strain behavior typical of unmodified bone grafts, while modified bone grafts will exhibit stress-strain behavior typical of similarly modified bone grafts. Further, due to differences in the material properties of individual samples, a first modified bone graft can exhibit stress-strain behavior that differs slightly from the stress-strain behavior of a second, but similarly modified, bone graft. Thus, the stress-strain curve of the first modified bone graft can be graphically represented on a stress-strain curve specific to that bone graft, and that stress-strain curve may differ slightly from the stress-strain curve of the second similarly modified bone graft. One of skill in the art will understand, however, that such differences can be statistically insignificant, such that modifying a number of bone grafts similarly, testing the similarly modified bone grafts, and averaging the resultant stress-strain data can yield a stress-strain curve typical of bone grafts modified in the same way.

FIG. 2 illustrates one embodiment of a stress-strain curve obtainable by modifying and testing a plurality of bone grafts. Modified bone grafts are first tested to obtain stress-strain data. The stress-strain values are then averaged and plotted to create a stress-strain curve that can be typical of bone grafts modified under the same or similar conditions. Persons of skill in the art will understand that the larger the number of samples tested, the more representative the average stress-strain values will be of other, untested samples.

As illustrated in FIG. 2, modified bone grafts exhibit a linear stress-strain relationship up to a yield point 50, which corresponds to a specific yield strength S_{y} (55). The linear portion 60 of the curve is known as the elastic deformation region. The slope of this linear portion corresponds to the modulus of elasticity or the modulus of compression, two of the material properties that can be optimized using modulus-matching methods described herein. The modulus of elasticity represents a material's tendency to be deformed elastically, or non-permanently. The yield strength, another material property that can be optimized, represents the stress at which the bone graft begins to deform plastically under the applied stress. Prior to reaching the specific yield strength S_{y} (55), the bone graft will deform elastically and will return to its original shape when the applied stress is removed. Once testing passes the yield strength, some amount of deformation in the bone graft will be permanent and non-reversible.

As deformation continues along the stress-strain curve in FIG. 2, the stress increases until the bone graft reaches the ultimate point 65, which corresponds to an ultimate strength 70. When the ultimate point is reached, during either tensile or compressive testing, the bone graft becomes unstable and ruptures or fractures. Ultimate strength 70 is another material property that can be optimized using modulus-matching methods described herein.

Additional material properties can be calculated using the stress-strain curve illustrated in FIG. 2. The resilience of the modified bone grafts can be calculated by determining the area of triangular region 75 under the elastic deformation region of the curve. Resilience is the property of a material to absorb energy when it is deformed elastically and to have this energy recovered upon unloading. Thus, a more resilient material can absorb more energy before it exceeds its elastic limit at the specific yield strength S_{y} (55), and begins to deform plastically, or permanently. The toughness of the bone grafts can also be calculated by determining the area of the region 80 under the entire stress-strain curve. Toughness is defined as the energy a material can absorb before it fails. Thus, a tougher material can absorb more energy before it ruptures or fractures. Resilience and toughness are material properties that can be tested and optimized using modulus-matching methods described herein.

The stiffness of the modified bone grafts can also be measured and optimized using methods described herein. Stiffness is the resistance of an elastic body to deformation by an applied force. Stiffness can be calculated by dividing the force (P) applied to a material by the displacement (δ) produced by that force. One measure of stiffness is obtained by dividing the load at yield by the displacement at yield.

Another material property that can be tested and optimized using methods described herein is the compressive strength of modified bone grafts. The compressive strength of a material can be calculated in a compressive test, during which a uniaxial compressive load is applied to a material, as opposed to the uniaxial tensile load used in a tensile test. As described above with reference to the tensile test results illustrated in FIG. 2, a stress-strain curve can be plotted using the stress and strain values of the modified bone grafts during a compressive test. The compressive strength of a material is defined as the value of uniaxial compressive stress reached when the material fails completely. Thus, compressive strength can be calculated by dividing the uniaxial compressive load at the point of failure by the cross-sectional area of the material at the point of failure. In addition, the slope of the linear portion leading up to the yield point is known as the modulus of compression. Thus, one measure of the modulus of compression is the stress at yield divided by the strain at yield during a compressive test.

Referring again to FIG. 1, the modification process at step 500 can be adjusted to optimize the material properties of a bone graft to more closely match the material properties of the patient's recipient bone. Material properties that can be optimized include but are not limited to yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis. Optional mechanical testing such as that described with reference to step 800 can then yield data on specific material properties of a modified bone graft. A comparison of the bone graft data to similar values obtained during mechanical testing of a sample of the patient's recipient bone can also be performed to ensure the modification process successfully matched the material properties of the bone graft and recipient bone as desired. In an optional step 900, the bone graft can then be freeze-dried for transportation to the site of implantation. At step 950, the modified bone graft can be rehydrated and implanted into the patient's recipient bone.

In some embodiments, the modification process at step 500 is performed on a portion of a patient's bone to modify its material properties as described above. The modified patient bone can then be implanted back into the patient.

Persons of skill in the art will understand that steps 100 to 400 described with reference to FIG. 1 can be performed by one entity, while the remaining steps 500 to 950 can be performed by a second entity. The methods and systems described herein for modulus matching a bone graft to a patient's bone do not require harvesting, cleaning, and machining of a bone graft, and can include obtaining a suitable bone graft from a conventional source, such as but not limited to a tissue bank.

The process described with reference to FIG. 1 can begin with a step for obtaining patient information and/or information on the patient's specific bone characteristics such as, but not limited to, age, gender, diagnosis, hormone profile, and BMD. Alternatively, obtaining patient information and/or bone information can take place at an intermediate step in the described process. Obtaining patient data could involve sampling a piece of patient bone and testing it to gather material property data. In one embodiment, obtaining patient data includes receiving patient data, entering that data into a database, and determining that the patient falls into one of a number of bone “type” categories. Each “type” of bone is expected to exhibit certain material properties based on experimentation and mechanical testing of numerous bone samples. The database can be developed by a doctor based on a number of diagnoses he has performed. Alternatively, a database can be developed as a result of sampling a number of patients' bones, testing the samples for their material properties, and grouping the bone samples by their material properties. For example, such testing can reveal that patients with bone mass densities within a range A typically exhibit a resilience within range X. The testing can also further reveal that patients with bone mass densities within range B typically exhibit a resilience within range Y. When a patient's data and/or bone characteristics are obtained or received, the data can be entered into the database to determine, for example, that the patient's bone possesses a resilience within range X. A verification step is optionally performed to verify the patient's bone has a resilience within range X.

Patient data can be obtained through various tests including, but not limited to, bone mass density tests, bone calcium percentage tests, hard mineral content tests, regular CT scanning, quantitative CT scanning (qCT), dual energy X-ray absorbtiometry (DEXA), micro CT scanning, and MRI scanning.

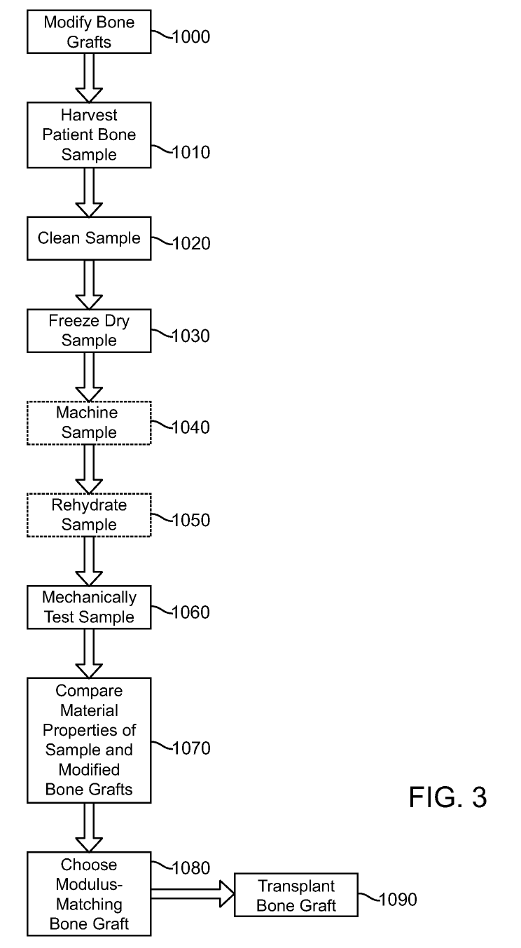

The skilled artisan will understand that embodiments of the present invention include modulus-matching methods that do not require mechanical testing of a modified bone graft. Another process for modulus-matching grafts in accordance with some embodiments of the present invention will now be described with reference to FIG. 3. The modulus-matching process begins at step 1000, where batches of bone grafts are modified in accordance with specific procedures to yield bone grafts having specific material properties, or material properties within a specific range of values. For example, a first series of bone grafts can then be demineralized in an acid wash having a first concentration for a first duration, resulting in bone grafts having a resilience within a first range of values. A second series of bone grafts can be demineralized in an acid wash having the first or a second concentration for a second duration, resulting in bone grafts having a resilience within a second range of values. The first and second ranges of values may or may not overlap. In addition, samples from these batches can be tested to confirm the demineralization process yields bone grafts exhibiting material properties within the desired range. The modified bone grafts can be freeze-dried after modification, and stored for extended periods of time, or until such time as a specific bone graft having resilience within a specific range of values is requested and matched with a specific patient's recipient bone having a specific resilience. The matched bone graft can then be transported to the surgical site, optionally rehydrated, then transplanted into the patient.

As used herein, “matching” a material or other property of a bone graft to the same property or properties in a patient's recipient bone means that the recipient bone's specific material characteristic falls within a range of values a modified bone graft is confirmed or expected to have. The range of values represents an improvement over or optimization of the modified bone graft's material property over that which would be exhibited by an unmodified bone graft. For example, an unmodified bone graft can exhibit a yield strength between 104 and 121 MPa. If a patient's recipient bone is tested and determined to have a yield strength of 85 MPa, a modified bone graft confirmed or expected to have a yield strength of between 80 and 90 MPa is a “match” for the recipient bone as defined herein. This is because the yield strength of the recipient bone, 85 MPa, falls within a range of yield strengths, 80 to 90 MPa, the bone graft is confirmed or expected to exhibit. Further, the confirmed or expected yield strength of between 80 and 90 MPa represents an improvement over or optimization of an unmodified bone graft exhibiting a yield strength of between 104 and 121 MPa. Thus, persons of skill in the art will understand that, in the above example, a modified bone graft need not be confirmed or expected to have a yield strength identical to that of the patient's recipient bone, 85 MPa, in order to be considered a match for the recipient bone.

The modulus-matching process according to some embodiments of the present invention continues at step 1010, where a surgeon harvests a sample of a patient's recipient bone. At step 1020, the sample is cleaned and at step 1030, the sample is freeze-dried. The patient sample is then transported to a local or remote location where mechanical testing of the sample can be performed. Prior to mechanical testing, the sample can optionally be machined into a cylindrical or other shape at step 1040. In addition, at step 1050, the patient sample can optionally be rehydrated prior to mechanical testing.

At step 1060, mechanical testing, including but not limited to tensile and compressive tests, are performed on the sample. Testing to determine the bone mass density and percentage of calcium can also be performed at step 1060. At step 1070, the material properties of the patient sample are compared to the material properties of the various modified bone grafts created at step 1000. Continuing to step 1080, a modified bone graft is selected that matches the known material properties of the patient sample. Persons of skill in the art will understand that two or more batches of bone grafts can be modified to exhibit certain material properties. For example, in some embodiments of the present invention, as many as eight groups of bone grafts are prepared at step 1000, each group of bone grafts exhibiting material properties within a specified range. Persons of skill in the art will also understand that a customized bone graft having desired material properties can be created at step 1070, as opposed to choosing a bone graft from one of the bone grafts prepared and stored at step 1000. In such a case, the desired modification can proceed as described with reference to step 500 in FIG. 1.

Once a modified bone graft is matched to the patient recipient's bone at step 1080, the modulus-matched bone graft is transported locally or remotely to the surgical site. At step 1090, the modulus-matched bone graft is implanted into the patient's recipient bone.

The modulus-matching process described herein includes methods of treating patients who suffer from a condition requiring a bone graft, wherein the patient's bones are ill-suited for graft implantation because the implant can cause end plate fracture in the patient's bone or implant subsidence due to significant differences between the modulus of elasticity, modulus of compression, and other material properties of the conventional graft and that of the recipient bone. Such a condition can include, for example, osteoporosis, aging spine pathologies, scoliosis, spinal degeneration, spondylolysis, spondylolisthesis, tumor, trauma, and infection. One aspect of the present invention includes a method of performing a spine intervention on a patient identified as requiring a bone graft. In a preferred embodiment, an osteoporotic patient is first identified. A bone graft is then harvested and optionally, cleaned and machined. The graft is next modified to more closely correlate to the patient's bone characteristics. The graft is then implanted into the osteoporotic patient's bone. By modifying the patient's bone graft and matching the graft more closely with the osteoporotic patient's bones, the risk of fracture and even failure in the recipient bone over time is greatly reduced. Persons of skill in the art will understand methods of treatment described herein are not limited to osteoporotic patients, and can be used in any patient that has been identified as requiring a bone graft.

Experiment 1

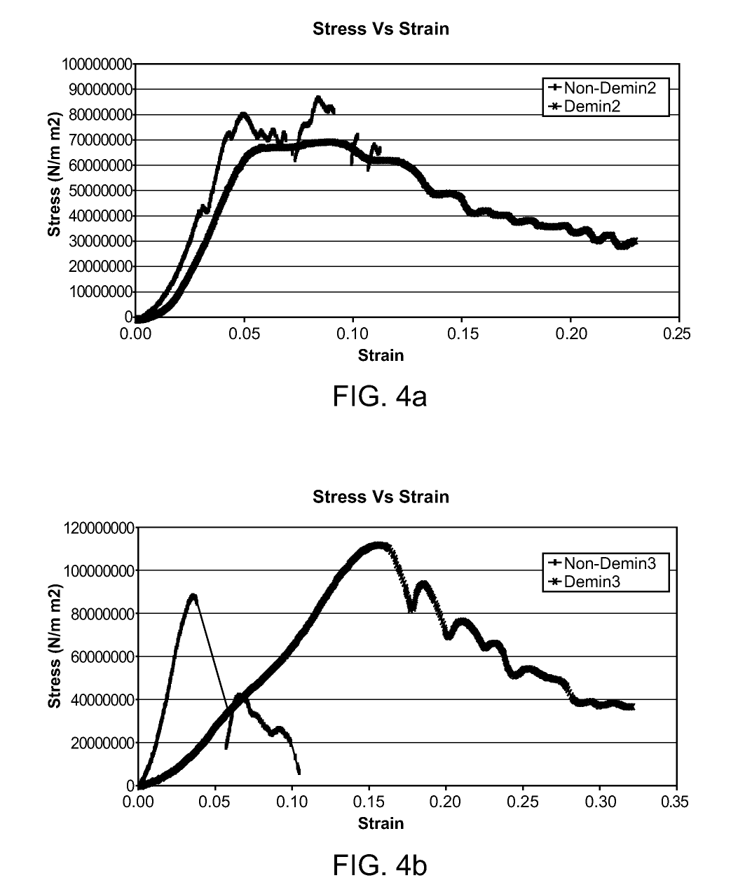

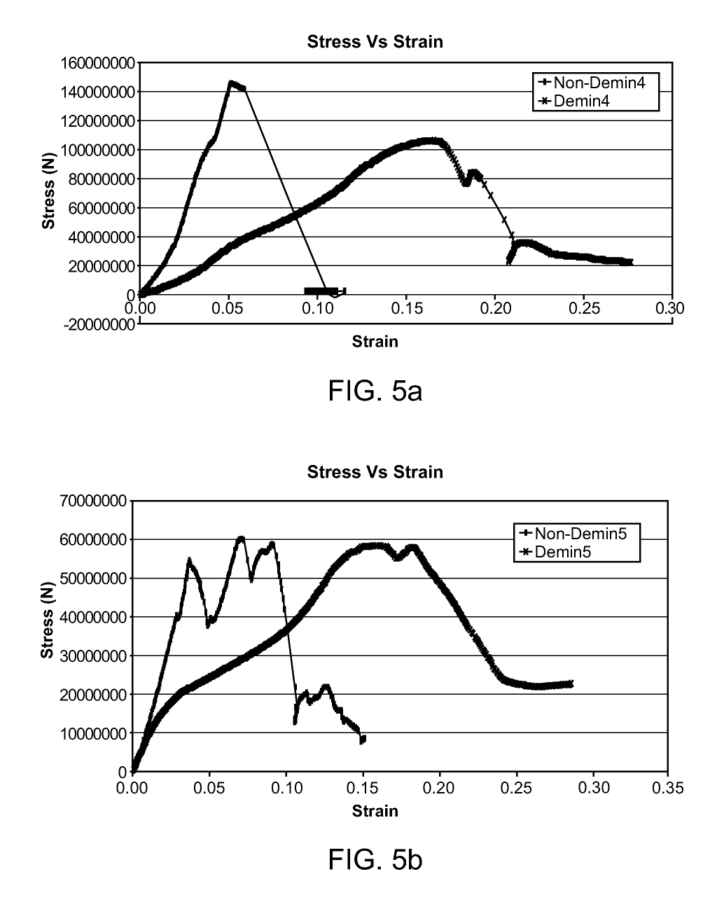

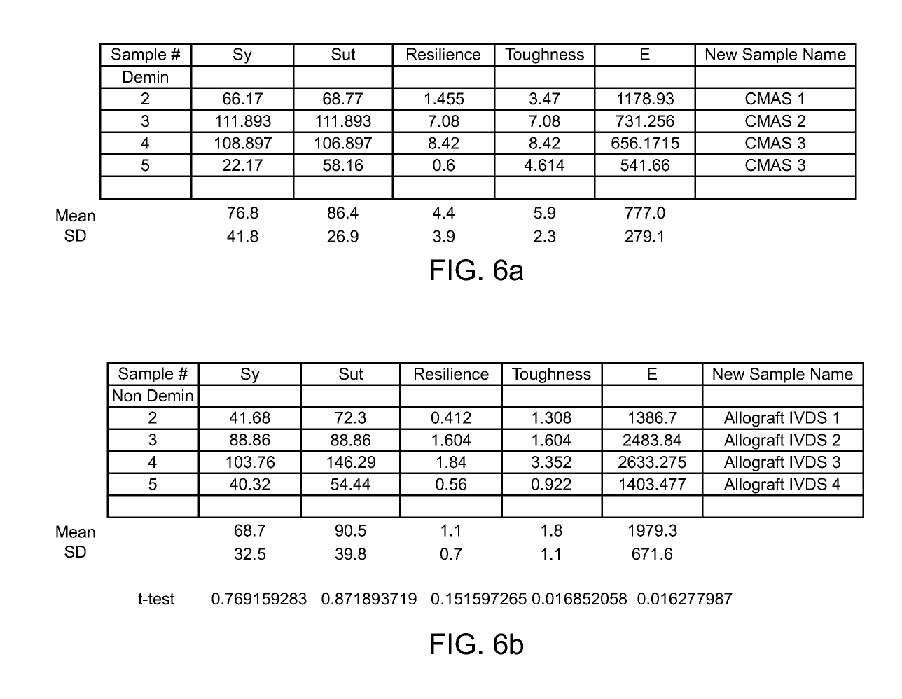

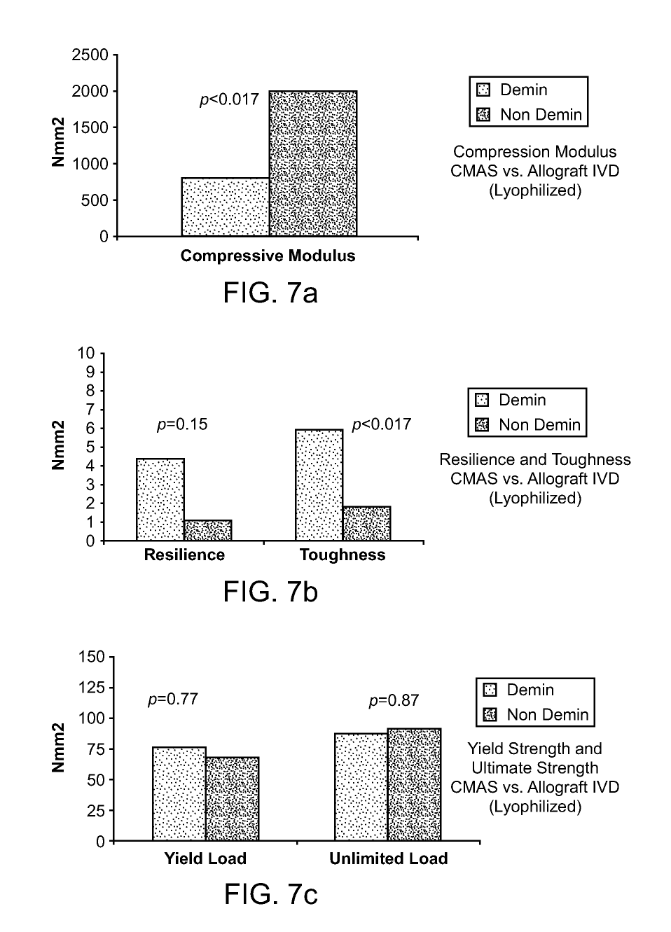

An experiment was conducted using donor bone tissue. Bone was harvested from a donor, cleaned, then machined into four anterior cervical allograft spacers. Each spacer was cut in half, one half being used as a control, the second half being used for testing and comparison to the control. All pieces were freeze-dried, removing substantially all moisture from the pieces. The test pieces were then placed on an orbital shaker and acid washed in solution of half-normal (0.5 N) hydrochloric acid for approximately 50 hours. The test pieces were then freeze-dried a second time. All control and test pieces were compression tested at a rate of 5 mm/min along the sagittal axis using a servo-hydraulic load frame. Stress and strain data for the test and control pieces was measured and plotted on stress-strain curves illustrated in FIGS. 4 and 5. Yield strength, ultimate strength, resilience, toughness and compressive modulus were calculated for the control pieces and the test pieces using methods described above. Mean values and standard deviation were also calculated. FIG. 6 summarizes these results. FIG. 7 compares the compressive modulus, resilience, toughness, yield strength, and ultimate strength of the control (or “Allograft IVD”) and test (or “CMAS”) pieces.

A comparison of the mean material property values of the test pieces and the control pieces demonstrates there was no statistically significant change in the yield strength or the ultimate strength of the test pieces as a result of demineralization. Notably, the resilience and toughness of the demineralized test pieces were greater than that of the control pieces. As described above, a more resilient material can absorb more energy before it exceeds its elastic limit and begins to deform permanently. Similarly, a tougher material can absorb more energy before it ruptures or fractures. Thus, the increase in resilience and toughness of the test pieces indicates the modified test pieces, on average, could absorb more energy before they exceeded their elastic limit, and could also absorb more energy before mechanical failure.

These surprising and unexpected improvements in the resilience and toughness of the modified test pieces indicate demineralization can improve material properties in bone grafts. Further, the demineralization process can be further optimized to generate a bone graft exhibiting material properties within a specific improved range. The modified bone graft with known material properties can then be matched to the specific characteristics of a patient recipient's bone before implantation. This optimization process can allow modulus-matching grafts to be implanted into a patient's bone, improving the interface between the graft bone and recipient bone and reducing the risk of fracture in the recipient bone over time.

Without being bound to any particular theory, it is believed that demineralizing the bone graft improves the resilience and toughness of the surface of the bone graft, resulting in a improved match between the material properties of the bone graft and the patient's recipient bone. Further, it is believed that subjecting the bone graft to varying acid concentrations and exposure times can alter the resilience and toughness characteristics of the bone graft as a whole, not just the surface properties of the bone graft.

Experiment 2

A similar experiment was performed using rehydrated bone tissue. As in Experiment 1, bone was harvested from a donor, cleaned, then machined into four anterior cervical allograft spacers. Each spacer was cut in half, one half being used as a control, the second half being used for testing and comparison to the control. All pieces were freeze-dried, removing substantially all moisture from the pieces. The test pieces were then placed on an orbital shaker and acid washed in solution of half-normal (0.5 N) hydrochloric acid for approximately 8 hours. The test pieces were then freeze-dried a second time.

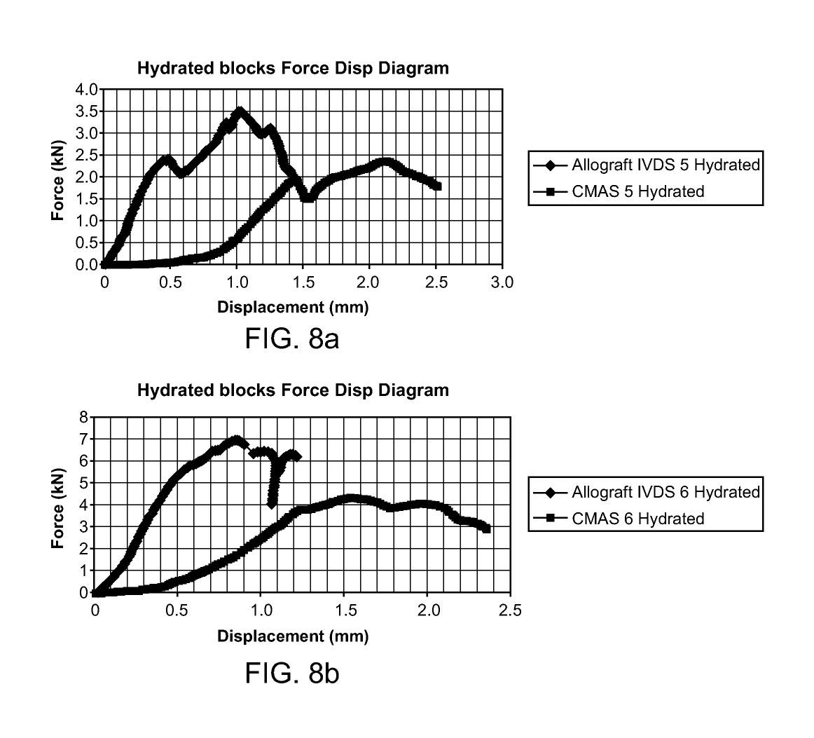

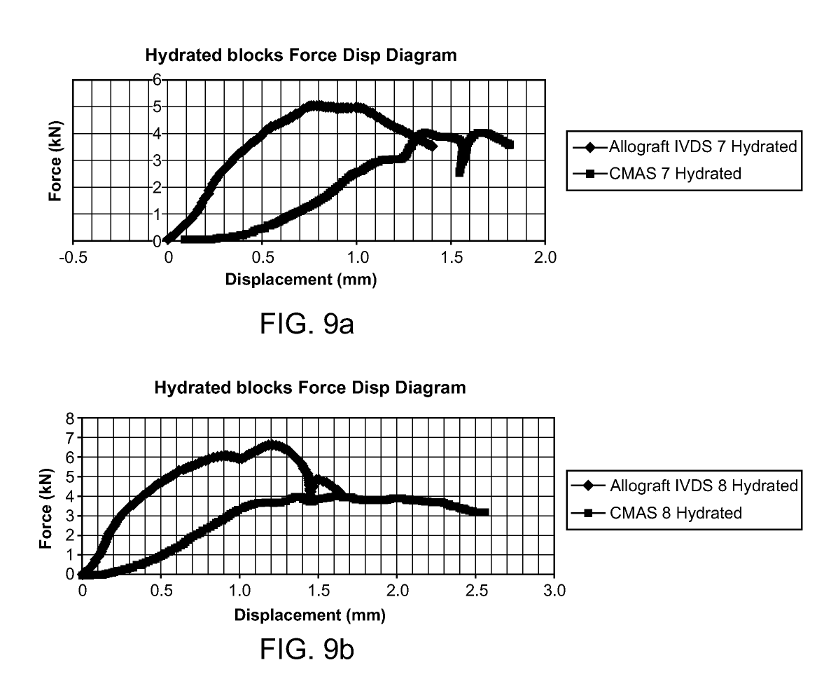

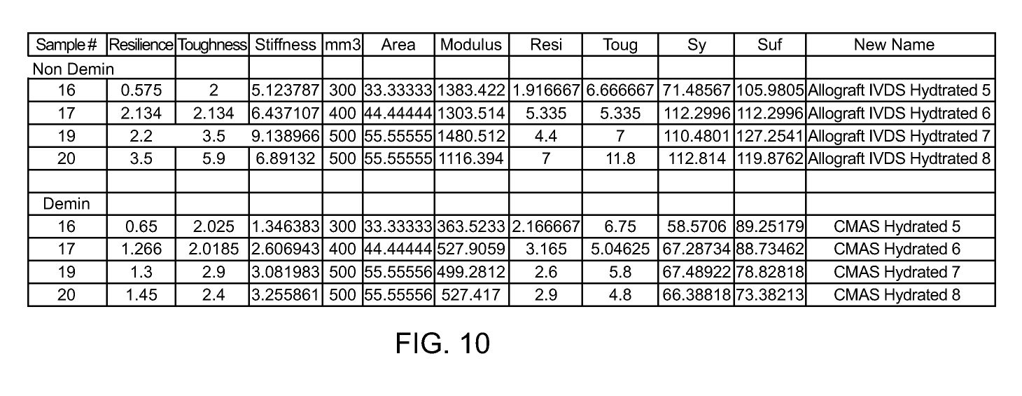

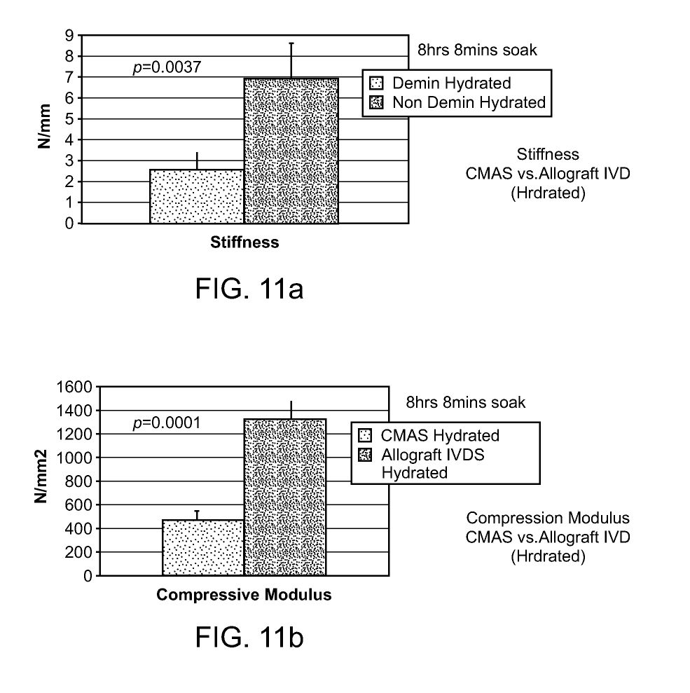

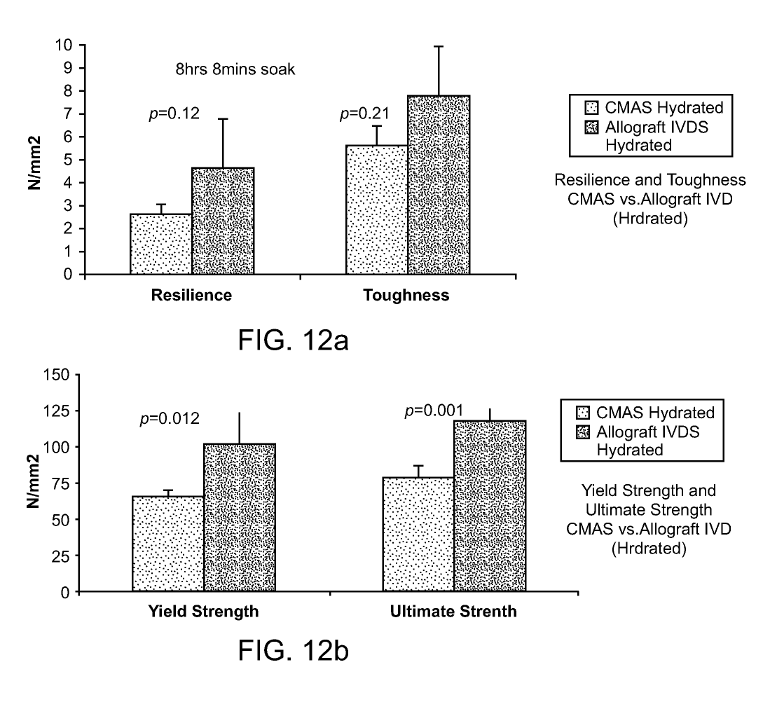

In this second experiment, all pieces were rehydrated in a saline solution before mechanical testing to more closely model common implantation practice in the surgical setting. The pieces were rehydrated for between approximately 1 to 1.5 hours. All control and test pieces were compression tested at a rate of 5 mm/min along the sagittal axis using a servo-hydraulic load frame. Force and displacement data for the test and control pieces was measured and plotted on graphs illustrated in FIGS. 8 and 9. Yield strength, ultimate strength, resilience, toughness, stiffness, and compressive modulus were calculated for the control pieces and the test pieces using methods described above. FIG. 10 summarizes these results. Mean values were also calculated. FIGS. 11 and 12 compare the stiffness, compressive modulus, resilience, toughness, yield strength, and ultimate strength of the control (or “Allograft IVD”) and test (or “CMAS”) pieces.

Summary of Results from Experiment 1 and 2

The test pieces in Experiment 1 and 2 showed reduced compressive modulus as compared to the control pieces. The test pieces in Experiment 1 and 2 also showed statistically similar yield strength, ultimate strength, resilience and toughness values as compared to the control pieces in both experiments. Thus, it may be possible that the reduced stiffness can lower the likelihood of endplate fractures and implant subsidence in patients with low bone density.

The present invention may also be provided as a bone graft kit comprising the modified bone graft discussed above and a vacuum sealed package, where the modified bone graft is sealed inside the package.

The present invention also discloses a method of treating an osteoporotic patient comprising identifying an osteoporotic patient; obtaining a bone graft; modifying the bone graft to more closely match the patient's bone characteristics; and implanting the bone graft into the osteoporotic patient.

It is to be understood that the detailed description and specific examples, while indicating preferred embodiments of the present invention, are given by way of illustration and not limitation. Many changes and modifications within the scope of the present invention can be made without departing from the spirit thereof, and the invention includes all such modifications.

What is claimed is:

1. A method of matching properties of a recipient bone with properties of a bone graft, the method comprising:
obtaining data on one or more material properties of a recipient bone;
comparing the material properties of the recipient bone to various modified bone grafts having differing range of values for the one or more material properties; and
selecting a modified bone graft having a range of values into which the one or more material properties of the recipient bone falls within.

2. The method of claim 1, wherein the one or more material properties include one of yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis.

3. The method of claim 1, wherein the one or more properties comprises one or more compositional properties of the recipient bone.

4. The method of claim 3, wherein the one or more compositional properties is one of bone mass density, bone calcium percentage, and hard mineral content.

5. The method of claim 1, further comprising freeze drying the modified bone graft.

6. A method of modifying the properties of a bone graft to match the properties of recipient bone, the method comprising:
obtaining data on one or more properties of a recipient bone;
modifying a bone graft to match the same one or more properties of the recipient bone;
testing the bone graft to confirm the one or more properties of the bone graft match the one or more properties of the recipient bone.

7. The method of claim 6, wherein obtaining data comprises harvesting and testing a sample of the recipient bone to determine one or more properties.

8. The method of claim 6, wherein the one or more material properties include one of yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis.

9. The method of claim 6, wherein the at least one or more properties comprises one or more compositional properties of the recipient bone.

10. The method of claim 9, wherein the one or more compositional properties is one of bone mass density, bone calcium percentage, and hard mineral content.

11. The method of claim 6, wherein modifying a bone graft comprises demineralizing the bone graft.

12. The method of claim 11, wherein demineralizing the bone graft comprises subjecting the bone graft to an acid wash.

13. The method of claim 11, wherein the bone graft is demineralized for between approximately 1 and 50 hours.

14. The method of claim 6, further comprising freeze drying the bone graft before testing the bone graft.

15. The method of claim 14, further comprising rehydrating the bone graft after freeze drying the bone graft and before testing the bone graft.

16. The method of claim 15, wherein rehydrating the bone graft comprises soaking the bone graft in one of saline, water, protein, blood, or bone marrow.

17. The method of claim 16, wherein the bone graft is soaked for between approximately thirty seconds to ninety minutes.

18. A method of modifying the properties of a bone graft to match the properties of recipient bone, the method comprising:
modifying a material property of a first bone graft, wherein the material property of the modified first bone graft falls within a first range of values;
modifying the same material property of a second bone graft, wherein the material property of the modified second bone graft falls within a second, non-overlapping range of values;
obtaining data on the same material property of a recipient bone; and
choosing the first or second bone graft, wherein the material property of the recipient bone falls within the range of material property values of the chosen bone graft.

19. The method of claim 18, wherein the one or more material properties include one of yield strength, ultimate strength (tensile, compressive, or shear), modulus of elasticity, Young's modulus, compression modulus, bulk modulus, rigidity modulus, shear modulus, compressive strength, resilience, toughness, stiffness, elastic energy, plastic energy, strain energy, and hysteresis.

20. The method of claim 18, wherein the at least one or more properties comprises one or more compositional properties of the recipient bone.
